(19) United States
(12) Patent Application Publication
(10) Pub. No.: US 2018/0271405 A1
(43) Pub. Date: Sep. 27, 2018

(54) Hyperglycemic Sensor Apparatus for Breath Gas Analysis

(71) Applicant: Spirosure, Inc., Pleasanton, CA (US)

(72) Inventors: Ryan R. Leard, Oakland, CA (US); David Steuerman, Silver Spring, MD (US); Solomon Ssenyange, Fremont, CA (US)

(73) Assignee: Spirosure, Inc., Pleasanton, CA (US)

(21) Appl. No.: 15/936,206

(22) Filed: Mar. 26, 2018

Related U.S. Application Data
(60) Provisional application No. 62/477,395, filed on Mar. 27, 2017.

(51) Int. Cl. A61B 5/08; A61B 5/097; G01N 33/497; G01N 33/98

(52) U.S. Cl. CPC A61B 5/082; A61B 5/097; G01N 2800/042; G01N 33/98; G01N 33/497

(57) ABSTRACT

A monitoring system is disclosed that includes features for detecting the presence of biomarkers from a gas sample, such as exhaled breath. An assembly includes a plurality of sensors to detect biomarkers present in exhaled breath that are associated with hyperglycemia. The biomarkers include, without limitation, acetone, ethanol, and methyl nitrate.

### CROSS REFERENCE TO RELATED APPLICATIONS

This application claims the benefit of U.S. Provisional Application No. 62/477,395 filed on Mar. 27, 2017, the subject matter of which is herein incorporated by reference in its entirety.

### FIELD OF THE INVENTION

The present invention relates generally to monitoring devices used for breath gas analysis, and more particularly to monitoring devices that may be used to test for biomarkers associated with medical conditions, such as hyperglycemia.

### BACKGROUND

Breath gas analysis can provide a method of providing information regarding the clinical state of an individual. Typically, a patient provides a breath sample generated from the act of exhalation, and one or more tests is performed on the exhaled breath gas sample. Breath gas analysis can be used to detect a wide range of compounds that are present in the blood and associated with certain medical conditions.

For example, hyperglycemia or high blood sugar is a condition in which high amounts of glucose are present in the blood. Diabetes mellitus is the most common cause of hyperglycemia, although other medical conditions may also cause elevated blood sugar levels. If left untreated, hyperglycemia can cause many serious complications, including the development of ketoacidosis, a condition in which the body does not have enough insulin. Thus, monitoring of blood glucose levels is important in the management of hyperglycemia and related medical conditions.

Using conventional methods, blood sugar levels may be measured by taking a blood sample from a patient's vein or from a small finger stick sample of blood. The test, however, involves an invasive technique that can sometimes cause discomfort and inconvenience. Analysis of exhaled breath is one potential alternate method of estimating glucose levels in the blood. The analysis presents challenges in that it requires high sensitivity to detect relatively small amounts of specific gases that are indicative of high blood sugar levels in the blood. The analysis also requires discrimination against the various other molecules that are present in human breath.

Thus, it would be desirable and advantageous to provide an accurate and efficient respiratory monitor capable of conducting tests for biomarkers, particularly multiple biomarkers associated with a particular condition or disease. It would also be desirable and advantageous to provide multiple sensors to detect diagnostic markers in an exhaled breath sample in a single unit or apparatus. In some instances, it also may be desirable and advantageous to provide a monitor with a compact and portable footprint that may be useful in a variety of settings, including in mobile health applications. Additionally, it would be desirable and advantageous to provide a detection system that is capable of detecting low concentrations of specific gases with high sensitivity, while discriminating against the various other molecules that may be present in human breath.

### BRIEF SUMMARY OF THE INVENTION

The present invention describes a solid-state sensor device, preferably a miniaturized solid-state sensor device, or a combination of sensor devices that can detect multiple gases in exhaled human breath.

In one embodiment, a solid-state sensor device or a combination of sensor devices that simultaneously detects at least three gases in exhaled breath is provided. The gases of interest include: ammonia, nitric oxide (NO), ethanol, acetone, methyl nitrate (or 2-, 3-pentyl nitrate), isoprene, carbon monoxide (CO), carbon dioxide (CO₂), propionic acid (or butanoic acid), aniline, o-toluidine, cyclopentane and 1-methyl-3-(1methylethyl)-benzene (CAS:535-77-3). Information regarding the use of an NO sensor in the detection of hyperglycemia may be found in Chenhu Sun, G. Maduraiveeran, Prabir Dutta; Nitric oxide sensors using combination of p- and n-type semiconducting oxides and its application for detecting NO in human breath; Sensors and Actuators B: Chemical 186 (2013) 117-125. In a preferred embodiment, the sensor device detects at least three of the above gases at concentrations ranging from 0 to 999 parts per billion (ppb), with discrimination against the hundreds of other molecules present in human breath.

Also described is an assembly for use in the detection of hyperglycemia, comprising a breath flow pathway; a breath inlet positioned at an entrance of the breath flow pathway; an ethanol selective sensor positioned in the breath flow pathway, downstream from the breath inlet; and a breath outlet positioned at an exit of the breath flow pathway, downstream from the ethanol selective sensor. In some embodiments, the ethanol selective sensor comprises a zinc oxide (ZnO) material deposited on a gold microelectrode array and a catalyst material.

Further described is an assembly for use in the detection of hyperglycemia comprising a breath flow pathway; a breath inlet positioned at an entrance of the breath flow pathway; a methyl nitrate selective sensor positioned in the breath flow pathway, downstream from the breath inlet; and a breath outlet positioned at an exit of the breath flow pathway, downstream from the methyl nitrate selective sensor. In some embodiments, the methyl nitrate selective sensor comprises a material adapted to catalyze the formation of nitrogen dioxide (NO₂) from methyl nitrate, a catalytic filter adapted to convert NO₂ to NO, and a sensor adapted to determine the concentration of NO.

A multi-sensor apparatus for use in the detection of hyperglycemia also is described. The apparatus comprises a housing comprising a breath flow pathway within the housing; a breath inlet positioned at an entrance of the breath flow pathway; a plurality of sensors positioned in the breath flow pathway, downstream from the breath inlet, wherein each sensor is configured to detect the presence of a biomarker indicative of hyperglycemia; and a breath outlet positioned at an exit of the breath flow pathway, downstream from the sensors. In some embodiments, the biomarker may be selected from the group consisting of acetone, ethanol, and methyl nitrate. Each sensor may be selected from the group consisting of an acetone selective sensor, an ethanol selective sensor, and a NO₂ selective sensor. The plurality of sensors may comprise an acetone selective sensor, an ethanol selective sensor, and a NO₂ selective sensor.

In addition, a method for detecting hyperglycemia comprises the steps of: flowing a breath gas sample into a housing comprising a breath flow pathway within the housing; flowing the breath gas sample through a breath inlet positioned at an entrance of the housing; exposing at least a portion of the breath gas sample to a plurality of sensors positioned in the breath flow pathway, downstream from the breath inlet; and releasing at least a portion of the breath gas sample through a breath outlet positioned at an exit of the breath flow pathway. Each sensor is configured to detect the presence of a biomarker indicative of hyperglycemia.

In some embodiments, an apparatus for detecting hyperglycemia may include a housing, a breath gas inlet, an acetone selective sensor element, an ethanol selective sensor element, a methyl nitrate selective sensor element, and a breath outlet. The housing comprises a breath flow pathway disposed within the housing. The breath gas inlet is positioned at an entrance of the breath flow pathway. The acetone selective sensor element is positioned in the breath flow pathway, downstream from the breath gas inlet. The ethanol selective sensor element is positioned in the breath flow pathway, downstream from the acetone selective sensor element. The methyl nitrate selective sensor element is positioned in the breath flow pathway, downstream from the ethanol selective sensor element. The breath outlet is positioned at an exit of the breath flow pathway, downstream from the sensor elements.

In some instances, the apparatus may further include a humidity controller configured to regulate the humidity of a breath sample in at least of a portion of the breath flow pathway. An excess exhaust portal may be adapted to release from the housing a portion of a breath gas sample entering the breath gas inlet, while another portion of the breath gas sample proceeds to the acetone selective sensor. The ethanol selective sensor element may comprise a zinc oxide material deposited on a gold microelectrode array and a catalyst material. The methyl nitrate selective sensor element may comprise a micro-channel reactor filter comprising platinum and zeolite, and a potentiometric NO sensor. The methyl nitrate selective sensor element may further comprise a micro-channel reactor filter heater relay.

In another embodiment, an apparatus for detecting hyperglycemia includes a housing comprising a breath flow pathway within the housing and a breath inlet positioned at an entrance of the breath flow pathway. The apparatus also includes a plurality of sensors positioned in the breath flow pathway, downstream from the breath inlet, and each sensor is configured to detect the presence of a biomarker indicative of hyperglycemia. A breath outlet is positioned at an exit of the breath flow pathway, downstream from the sensors. In some embodiments, the plurality of sensors includes an acetone selective sensor, an ethanol selective sensor, and a methyl nitrate selective sensor. The plurality of sensors may be selected from the group consisting of an acetone selective sensor, an ethanol selective sensor, and a methyl nitrate selective sensor. The biomarker may be selected from the group consisting of acetone, ethanol, and methyl nitrate.

Also described is a method for detecting hyperglycemia that includes the steps of flowing a breath gas sample through a breath inlet positioned at an entrance of a housing, and exposing the breath gas sample to an acetone sensor element positioned in the breath flow pathway, downstream from the breath inlet. The housing comprises a breath flow pathway disposed within the housing. The method further includes the steps of exposing the breath gas sample to an ethanol sensor element positioned in the breath flow pathway, downstream from the acetone sensor element; and exposing the breath gas sample to a methyl nitrate sensor element positioned in the breath flow pathway, downstream from the ethanol sensor element. The breath gas sample is released through a breath outlet positioned at an exit of the breath flow pathway.

Further described is a method for monitoring hyperglycemia that includes flowing a breath gas sample through a breath inlet positioned at an entrance of a housing. The housing comprises a breath flow pathway disposed within the housing. The method also includes the step of exposing the breath gas sample to a plurality of sensors positioned in the breath flow pathway, downstream from the breath inlet. Each sensor is configured to detect the presence of a biomarker indicative of hyperglycemia. The breath gas sample is released through a breath outlet positioned at an exit of the breath flow pathway.

In some embodiments, a sensor may include multiple sensor units, each providing one or more signals that may be indicative of the presence or concentration of a particular analyte. The analyte may be detected, or its concentration estimated, based on the signals obtained from the multiple sensor units.

Fewer than the sensors in the above examples, additional sensors, different combinations, other sensors, or sub-combinations of the described sensors may be used for the detection of blood glucose levels. Moreover, the sensors and related components may be positioned in different configurations and breath flow pathways than those described in the above examples.

### BRIEF DESCRIPTION OF THE SEVERAL VIEWS OF THE DRAWINGS

FIGS. 1A through 1C illustrate schematically the use of catalysts on p-n junction device structures. FIG. 1A illustrates schematically in cross-sectional side view a catalyst deposited on a p-type region, in accordance with one embodiment of the present invention. FIG. 1B illustrates schematically in cross-sectional side view a catalyst deposited on an n-type region, in accordance with one embodiment of the present invention. FIG. 1C illustrates schematically in cross-sectional side view catalysts deposited on both p-type and n-type regions, in accordance with one embodiment of the present invention.

FIG. 2 illustrates schematically a top view of a p-n gas sensor using zinc oxide (ZnO) deposited on a substrate as the p-type material, in accordance with one embodiment of the present invention. The sensor may be used for the detection of methyl nitrate in a breath gas sample.

FIG. 3 illustrates schematically a top view of a p-n gas sensor using a catalyst deposited on a substrate as the p-type material, in accordance with one embodiment of the present invention. The sensor may be used for the detection of nitrogen dioxide (NO₂) sensor from methyl nitrate in a breath gas sample.

FIG. 4A illustrates schematically a side view of a breath gas sample contacting a catalyst filter used for the detection of methyl nitrate, in accordance with one embodiment of the present invention. FIG. 4B illustrates schematically a side view of the formation of nitric oxide (NO) from NO₂ at the sensor.

FIGS. 5A through 5C illustrate schematically a device that includes an acetone selective sensor, ethanol selective sensor, and NO₂ selective sensor for methyl nitrate, in accordance with one embodiment of the present invention. FIG. 5A illustrates schematically a perspective view from the back of the device. FIG. 5B illustrates schematically a perspective view from the front of the device. FIG. 5C illustrates schematically a cutaway view of internal components of the device.

### DETAILED DESCRIPTION OF THE INVENTION

As used herein, the terms “comprises,” “comprising,” “includes,” “including,” “has,” “having,” or any other variation thereof, are intended to cover a non-exclusive inclusion. For example, processes, methods, articles, or apparatuses that comprise a list of elements are not necessarily limited to only those elements but may include other elements not expressly listed or inherent to such processes, methods, articles, or apparatuses. Further, unless expressly stated to the contrary, “or” refers to an inclusive “or” but not to an exclusive “or.” For example, a condition A or B is satisfied by any one of the following: A is true (or present) and B is false (or not present), A is false (or not present) and B is true (or present), and both A and B are true (or present).

Also, use of “a” or “an” are employed to describe the elements and components of the invention. This is done merely for convenience and to give a general sense of the invention. This description includes one or at least one, and the singular also includes the plural unless it is obvious that it is meant otherwise.

Unless otherwise defined, technical and scientific terms used herein have the same meaning as commonly understood by one of ordinary skill in the art to which this invention belongs. Although methods that are similar or equivalent to those described herein can be used in the practice or testing of the present invention, suitable methods and materials are described herein. In case of conflict, the present specification, including definitions, will control. In addition, materials, methods, and examples are illustrative only and not intended to be limiting.

In the following description, numerous specific details, such as the identification of various system components, are provided to understand the embodiments of the invention. One skilled in the art will recognize, however, that embodiments of the invention can be practiced without one or more of the specific details, ordinary methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or work characteristics may be combined in any suitable manner in one or more embodiments.

In one embodiment, a sensor device uses a combination of p- and n-metal oxides deposited on a gold microelectrode array. The sensor device is designed so that a sensor has several leads or electrodes having different selective catalyst material on top. The catalyst materials are designed and printed so as to promote selectivity. The sensor devices are also designed so that the selective catalyst material serves as the catalyst filter to an incoming breath stream. Because the p-type and n-type semiconductors show reverse conductivity response to the test gases due to their opposite charge carriers, the combination of a p-n junction is beneficial to cancel signal from different analyte species.

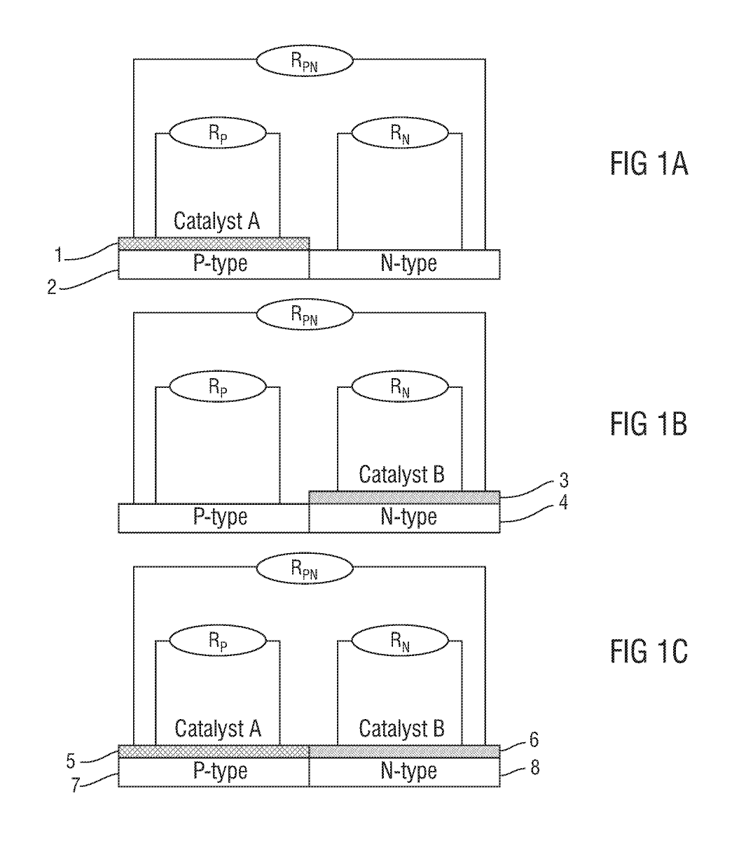

Referring to FIGS. 1A through 1C, a schematic diagram illustrating the use of catalysts on a p-n junction device structure is shown. The device structure includes a p-type semiconductor material on one side and an n-type semiconductor material on the other side. Referring to FIG. 1A, a catalyst material (Catalyst A) 1 may be deposited on the p-type region 2. Referring to FIG. 1B, a catalyst material (Catalyst B) 3 may be deposited on the n-type region 4. Referring to FIG. 1C, catalyst materials 5 and 6 (Catalyst A and Catalyst B) may be deposited on both the p-type 7 and n-type regions 8, respectively. As depicted in the figures, R_{N}, R_{P}, and R_{PN} are the resistances measured between the n-n, p-p, and p-n type regions, respectively. These resistances may provide information regarding the reactions occurring at the surface of the device. In some embodiments, instead of measuring resistances across one or more regions, a voltage may be applied and the current measured to detect reactions occurring at the surface of the device. For example, a current increase might indicate the reaction of certain molecules at the surface, while a current decrease might indicate less reaction of those molecules at the surface.

The catalyst material may be selected to react with specific compounds present in a gas sample and catalyze the formation of a particular species of interest. Non-limiting examples of suitable catalysts, particularly in the detection of compounds in breath gas that are associated with hyperglycemia, include nickel, gold, platinum, titanium, or other metals. For example, when a platinum catalyst is deposited on the p-type region, the platinum catalyst will selectively react with ammonia in terms of oxidation.

In some embodiments, treatment or modification of the surfaces and materials may enhance the selectively of the device. For example, increases in the available surface area of the device may increase the sensitivity of the device to a particular gas of interest. The thickness of the film material may be increased to increase the sensitivity. As another example, the multiple p-type regions may be treated in series. The surfaces may otherwise be treated to promote the catalyst reaction. In other embodiments, the p- or n-type materials may be doped with other ions. For example, an amount of potassium or sodium may be added to the n-type semiconductor to enhance its reactivity. Less may be added to decrease its reactivity. In addition, the resistivity of the material may be changed. A suitable resistivity may be low enough to be measured by the equipment, but not too low so as to cause interference with other elements of the device.

In some embodiments, the p-n junction in the sensor device may be designed to detect one or more of the compounds that are present in patients with hyperglycemia. Several gases have been reported in the literature to indicate the onset of diabetic hyperglycemia. These target compounds include, without limitation: ethanol, acetone, methyl nitrate, isoprene, cyclopentane, and 1-methyl-3-(1 methylethyl)-benzene. In preferred embodiments, the p-n junction in the sensor device may be designed to detect one or more of the following gases from the above list: acetone, ethanol, and methyl-nitrate. These gases, when measured in the parts-per-billion (ppb) range, are shown to increase when a person is exhibiting an increase in blood glucose levels. A discussion of the potential for using these exhaled compounds as diagnostic markers for glycemic markers can be found in Lisa L. Samuelson, David A. Gerber, M D; Recent Developments in Less Invasive Technology to Monitor Blood Glucose Levels in Patients with Diabetes; Labmedicine; Vol 40 Number 10 (2009) 607-610.

For example, in some embodiments, the present invention provides for an ethanol selective sensor using zinc oxide (ZnO) nanosheets. ZnO nanosheets have been successfully synthesized through a hydrothermal process, followed by annealing of the zinc carbonate hydroxide hydrate precursors. A description of the synthesis and ethanol response of hierarchically porous ZnO nanosheets is described in Lexi Zhang, Jianghong Zhao, Haiqiang Lu, Li Li, Jianfeng Zheng, Hui Li, Zhenping Zhu; Facile synthesis and ultrahigh ethanol response of hierarchically porous ZnO nanosheets; Sensors and Actuators B: Chemical; 161 (2012) 209-215. The ZnO nanosheets are single crystals with hexagonal wurtzite and mesoporous structures. Gas sensors based on these ZnO nanosheets exhibited ultrahigh response, fast response-recovery, and good selectivity for ethanol and stability to approximately 0.01-1,000 ppm (parts per million) ethanol at approximately 400° C. Low concentrations of ethanol (down to 10 ppb (parts per billion)), among the lowest detection limits for ethanol utilizing pure ZnO as sensing materials in a one-side heated gas sensor, can be readily detected (S=3.05+/−0.21).

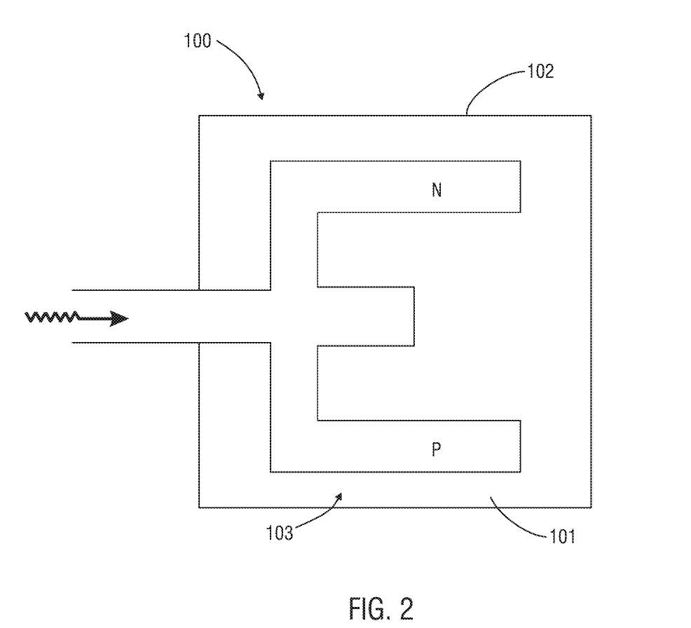

Referring to FIG. 2, a gas sensor 100 using ZnO 101 deposited on a substrate 102 as the p-type material may be used to detect ethanol in a breath sample. A catalyst (such as platinum, silver, gold, or palladium) is also used. Breath gas containing ethanol gas 103 flows over the substrate, where it may be detected. Other materials and catalysts may be used for the detection of other compounds of interest using the p-n junction device structure.

In another example, the present invention also provides a methyl nitrate selective sensor that uses a catalyst filter. The reaction of organic nitrates with various catalytic compounds has been studied. The catalysts fall into three categories. In some instances, they may have no effect on the rate of loss of organic nitrate or the type of products generated over the thermal base case. In other instances, the catalyst may accelerate the loss of the organic nitrate, but the products remain the same as the thermal base case. In yet other instances, the catalyst may accelerate the loss of organic nitrate and generate different products than the thermal base case. Consistent with the third category, it has been shown that catalytic amounts (3 mol %) of copper (II) oleate and iron (III) acetyl acetonate each increased the rate of loss of organic nitrates at 170° C. under nitrogen (N₂) by a factor of 1.5 and 2, respectively. The reactions remained first order (or pseudo-first order). The reaction of organic nitrates with these catalytic compounds are discussed in M. A. Francisco* and J. Krylowski; Chemistry of Organic Nitrates: Thermal Oxidative and Catalytic Chemistry of Organic Nitrates; Energy and Fuels; 24 (2010) 3831-3839.

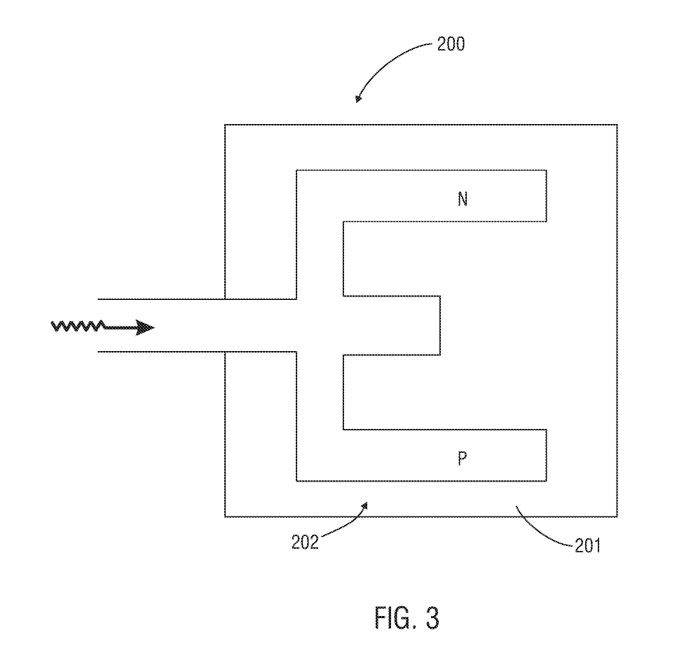

Referring to FIG. 3, a gas sensor 200 with a p-n type junction on an electrode is shown. A catalyst, such as copper (II) oleate, iron (III) acetyl acetonate, or molybdenum (II) dithiocarbamate, is deposited on the substrate 201. As breath gas containing methyl nitrate (CH₃NO) 202 flows across the reactor plate, the catalyst facilitates the formation of nitrogen dioxide (NO₂) from the methyl nitrate.

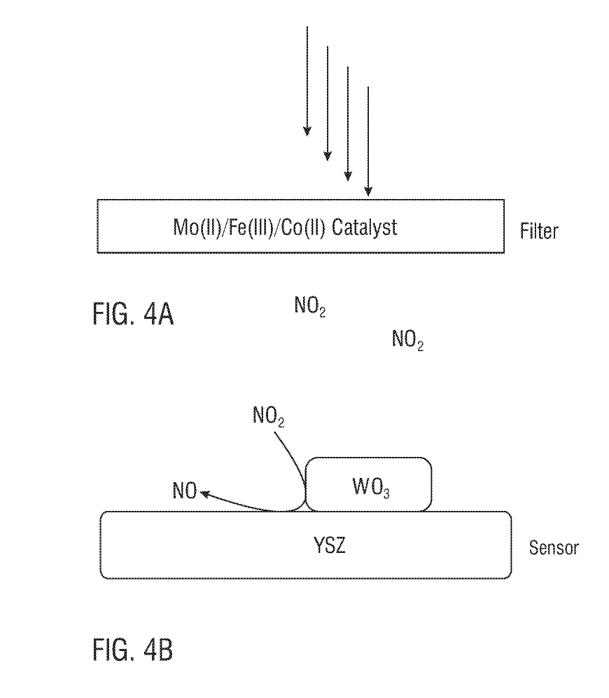

As depicted in FIG. 4A, the catalyst acts as a converter for the selective formation of a specific mixture of nitric oxide (NO) and NO₂ from the methyl nitrate. The mixture of NO and NO₂ is then directed to a sensor that includes working electrode tungsten oxide (WO₃) on a solid electrolyte yttria-stabliized zirconia (YSZ). At the sensor, NO₂ is reduced to NO and a signal is detected during the electron transfer process at the air, WO₃ and YSZ triple-point, as depicted in FIG. 4B. In a preferred embodiment, the breath gas is then exposed to a catalytic filter in a micro-channel reactor with spaces that allow for gas flow through a compact structure adapted to convert NO₂ to NO. A sensor may then be used to determine the concentration of NO in the breath gas. The measured concentration of NO then may be correlated with the methyl nitrate concentration present in the exhaled gas sample.

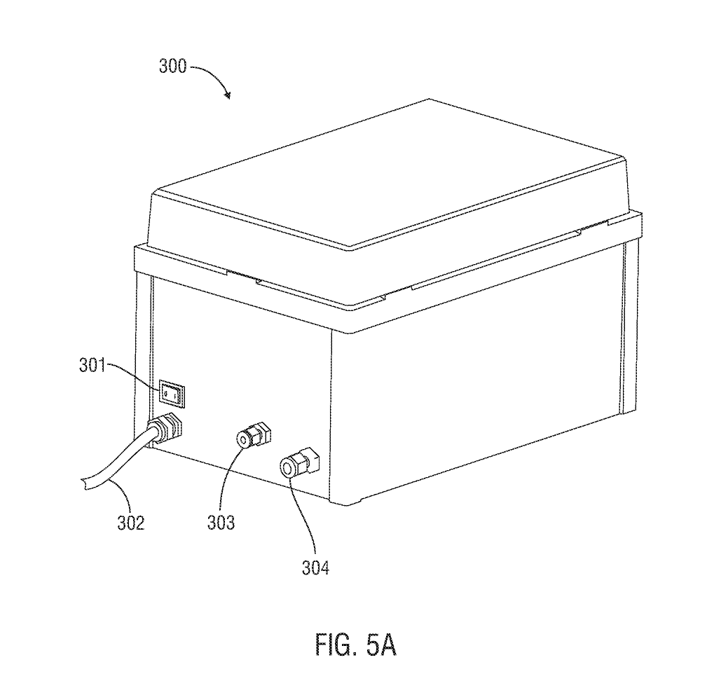

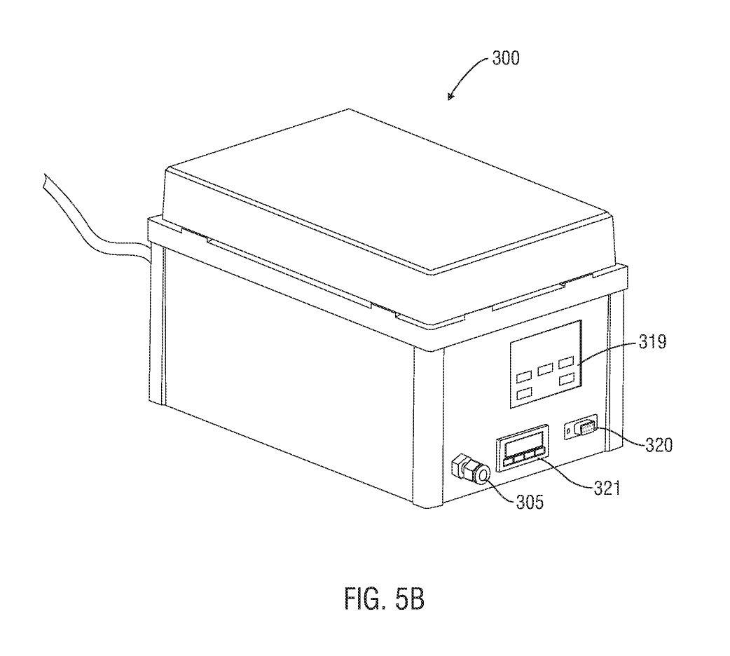

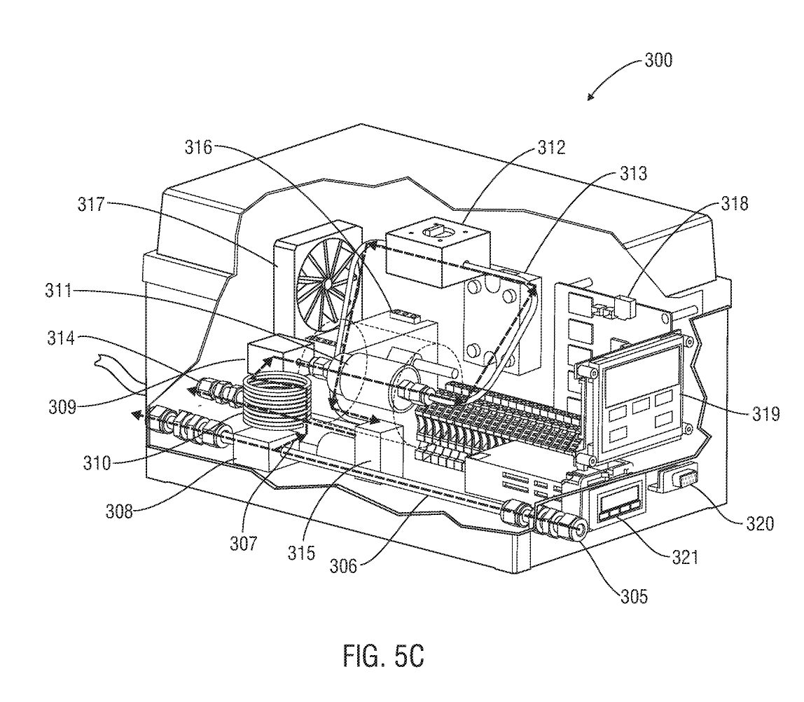

The sensors may be used alone, in combination with, or in conjunction with other types of sensors in a single unit to allow for the detection of multiple gases of interest from breath gas analysis. Referring to FIGS. 5A through 5C, an apparatus 300 for the detection of hyperglycemia, according to one embodiment of the present invention, is shown. As shown in FIG. 5A, on one side of the enclosure is a power switch 301, an A/C power cord 302, an outlet for breath exhaust 303, and an excess breath exhaust outlet 304 that forms a secondary outlet for breath gas flow. As shown in FIG. 5B, breath gas enters a breath inlet 305 on the other side of the enclosure. As shown in FIG. 5C, a portion of breath gas exits the system through a flow pathway 306 that terminates at the excess breath exhaust portal without contacting the sensors, while the non-exhausted breath gas is channeled through various sensors. In a preferred embodiment, most of the breath gas exits the apparatus through the breath exhaust port. For example, approximately 95% or more of the breath gas volume is exhausted as excess breath, while approximately 5% of less of the breath gas volume proceeds through the apparatus for analysis.

In the embodiment shown in FIG. 5C, the analyzed gas flows through a breath flow pathway 307 that includes a series of sensors selected to detect certain diagnostic markers for hyperglycemia. The breath flow pathway includes an inlet positioned at an entrance of the breath flow pathway and an acetone selective sensor 308. The acetone selective sensor may be one or a combination of commercially available gas sensors that are known in the art for detecting the presence of acetone in a gas. The gas then proceeds through an ethanol selective sensor 309, such as a gas sensor using ZnO deposited on a substrate, with a catalyst such as platinum, silver, gold, or palladium, as described above. The described sensor is provided for non-limiting, illustrative purposes, and other types of sensors may be used to detect ethanol in a gas sample. The humidity of the gas may be controlled using one or more humidity controllers 310 to regulate the humidity of the breath gas flowing through the breath flow pathway. As an example, it may be desirable to control the humidity of the gas before it is channeled to and analyzed by an NO sensor assembly.

The breath gas is directed to a micro-channel reactor filter (MCR) 311 and sensor 312 for the detection of methyl nitrate. A micro-channel reactor filter heater relay 313 may also be included. In some embodiments, the methyl nitrate selective sensor may be configured to detect NO₂, as described above. The MCR and sensor assembly are configured to determine the total NO concentration from the breath sample gas. A patient's breath sample can include nitrogen oxides (NOₓ), which includes pure NO, pure NO₂, and mixtures thereof. The gas introduced from the patient's breath typically has concentrations of NO, NO₂ and carbon monoxide (CO) in the range of 0 to 1000 ppb. The MCR filter includes a catalyst filter comprising platinum and zeolite within a flow pathway. The gas flowing through the flow pathway interacts with the catalyst filter at a particular temperature to form an equilibrium mixture of NO and NO₂. The MCR and sensor assembly further includes a sensor element configured to sense the amount of NOₓ flowing therethrough. In a preferred embodiment, the sensor element includes two electrodes on a solid electrolyte yttria-stabilized zirconia as follows: (1) a sensing potentiometric electrode disposed downstream of the catalytic filter device so as to contact the equilibrium mixture of NO and NO₂, and (2) a reference potentiometric electrode. Because the relative amounts of NO and NO₂ are known due to the equilibrium reaction through the filter, the NOₓ reading of the sensor can be used to determine the amount of NO in the sample.

The sensor and the microchannel reactor are maintained at different temperatures to provide a driving force for the NOx equilibration reactions. That is, the reactor equilibrates the NO to NO₂ mixture based principally on the temperature of the reactor (which includes platinum-zeolite (PtY)), and then the potential develops on the sensor element based on this equilibration of NO and NO₂ changing when reacting with reference electrode (PtY) and the sensing electrode at a temperature different than the temperature of the reactor. The sensor works by measuring the potential difference between the two electrodes, and a total NOx concentration (and then NO concentration) can be calculated by comparing the potential to a calibration curve. Details regarding a reactor and sensor assembly are described in U.S. Patent Publication Nos. 2015-0250408-A1 and 2017-0065208-A1, both entitled “Respiratory Monitor,” the entirety of which are incorporated by reference herein. The measured concentration of NO then may be correlated with the methyl nitrate concentration present in the exhaled gas sample. Other types of sensors, however, also may be used to detect the presence of methyl nitrate in a breath gas sample.

The analyzed breath gas is then directed through the breath exhaust portal 314 using a pump 315. In this example, the breath exhaust portal forms a breath outlet positioned at an exit of the breath flow pathway. The apparatus also may include an A/C DC power supply 316 and a case fan 317 for cooling. Control and acquisition electronics 318, as well as an LCD touch screen 319 that allows a user to enter information may be included, as well. The apparatus may also include external communications output (wired or wireless) 320, along with an Omega temperature controller 321. In a preferred embodiment, the components of the apparatus are contained with an enclosure that measures approximately 7.5 inches in height, 7.7 inches in width, and 11.6 inches in length.

In some embodiments, information obtained from the sensors may be used to provide qualitative data for a patient whose breath gas has been analyzed. For example, the measurements obtained from the sensors may be used to determine whether a given patient may exhibits (or not exhibit) certain indicators of hyperglycemia. The information also may be used to obtain quantitative results, such as specific levels of certain gases in a patient's breath.

It will be appreciated that fewer than the sensors or additional sensors (e.g., different combinations, other sensors, or sub-combinations of the described sensors may be used for the detection of blood glucose levels. Moreover, the sensors and related components may be positioned in different configurations and breath flow pathways than those described in the above examples. For example, breath gas proceeding through the breath flow pathway may be exposed to the sensors in different sequences from those discussed above.

Breath gas proceeding through the breath flow pathway may be exposed to additional sensors, intermediate sensors, mechanical components, intermediate components, and other system components. For example, the system may include additional components to pre-condition or treat a given gas sample before being exposed to a sensor module. In addition, breath gas proceeding through the breath flow pathway may be exposed to additional sensors, intermediate sensors, mechanical components, intermediate components, and other system components through different pathways.

It also will be appreciated that each of the analytes indicative of hyperglycemia in a breath gas sample (e.g., ethanol, acetone, methyl nitrate, isoprene, cyclopentane, and 1-methyl-3-(1 methylethyl)-benzene, or other analytes) is not limited to detection by the p-n junction device structures described or the devices described in the above examples. That is, any sensor or combination of sensors that are capable of detecting the presence of the described analytes that are indicative of hyperglycemia in a breath sample may be used.

Each of the analytes indicative of hyperglycemia in a breath gas sample (e.g., ethanol, acetone, methyl nitrate, isoprene, cyclopentane, and 1-methyl-3-(1 methylethyl)-benzene, or other analytes) is not limited to detection by a single sensor. The sensors described above are not limited to single sensor assemblies, each providing a single signal. Rather, in some embodiments, a sensor may include multiple sensor assemblies, and each sensor assembly may provide its own signal or set of signals. The analyte or analytes of interest may be detected, or its concentration estimated, from signals obtained from the sensor assemblies.

It also is contemplated that a single apparatus may include sensors for the detection of multiple conditions from a given breath gas sample. For example, the apparatus may include sensors for detecting known biomarkers for respiratory diseases such as CO, carbon dioxide (CO₂), and/or NO, along with sensors for detecting known biomarkers for hyperglycemia such as ethanol, acetone, methyl nitrate, isoprene, cyclopentane, and 1-methyl-3-(1 methylethyl)-benzene. In this configuration, the apparatus would allow for the detection of respiratory diseases and hyperglycemia from a patient's breath sample.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with anyone or more of the features described herein. The breadth of the present invention is not to be limited by the subject specification, but rather only by the plain meaning of the claim terms employed.

This disclosure is sufficient to enable one of ordinary skill in the art to practice the invention, and provides the best mode of practicing the invention presently contemplated by the inventor. While a full and complete disclosure is made of specific embodiments of this invention, the invention is not limited by the exact construction, dimensional relationships, and operation shown and described. Various modifications, alternative constructions, design options, changes and equivalents will be readily apparent to those skilled in the art and may be employed, as suitable, without departing from the spirit and scope of the invention. Such changes might involve alternative materials, components, structural arrangements, sizes, shapes, forms, functions, operational features and the like.

What is claimed is:

1. An apparatus for detecting hyperglycemia comprising:
(a) a housing comprising a breath flow pathway disposed within the housing;
(b) a breath gas inlet positioned at an entrance of the breath flow pathway;
(c) an acetone selective sensor element positioned in the breath flow pathway, downstream from the breath gas inlet;
(d) a ethanol selective sensor element positioned in the breath flow pathway, downstream from the acetone selective sensor element;
(e) a methyl nitrate selective sensor element positioned in the breath flow pathway, downstream from the ethanol selective sensor element; and
(f) a breath outlet positioned at an exit of the breath flow pathway, downstream from the sensor elements.

2. The apparatus of claim 1, further comprising a humidity controller configured to regulate the humidity of a breath sample in at least a portion of the breath flow pathway.

3. The apparatus of claim 1, further comprising an excess exhaust portal adapted to release from the housing a portion of a breath gas sample entering the breath gas inlet while another portion of the breath gas sample proceeds to the acetone selective sensor.

4. The apparatus of claim 1, wherein the ethanol selective sensor element comprises a zinc oxide material deposited on a gold microelectrode array and a catalyst material.

5. The apparatus of claim 4, wherein the methyl nitrate selective sensor element comprises a micro-channel reactor filter comprising platinum and zeolite, and a potentiometric nitric oxide (NO) sensor.

6. The apparatus of claim 5, wherein the methyl nitrate selective sensor element further comprises a micro-channel reactor filter heater relay.

7. An apparatus for detecting hyperglycemia comprising:
(a) a housing comprising a breath flow pathway within the housing;
(b) a breath inlet positioned at an entrance of the breath flow pathway;
(c) a plurality of sensors positioned in the breath flow pathway, downstream from the breath inlet, wherein each sensor is configured to detect the presence of a biomarker indicative of hyperglycemia; and
(d) a breath outlet positioned at an exit of the breath flow pathway, downstream from the sensors.

8. The apparatus of claim 7, further comprising a humidity controller configured to regulate the humidity of a breath sample in at least a portion of the breath flow pathway.

9. The apparatus of claim 7, further comprising an excess exhaust portal, wherein the excess exhaust portal forms a secondary outlet for breath flow.

10. The apparatus of claim 7, wherein the plurality of sensors comprises an acetone selective sensor, an ethanol selective sensor, and a methyl nitrate selective sensor.

11. The apparatus of claim 10, wherein the ethanol selective sensor comprises a zinc oxide material deposited on a gold microelectrode array and a catalyst material.

12. The apparatus of claim 10, wherein the methyl nitrate selective sensor comprises a micro-channel reactor filter comprising platinum and zeolite, and a potentiometric nitric oxide (NO) sensor.

13. The apparatus of claim 12, wherein the methyl nitrate selective sensor further comprises a micro-channel reactor filter heater relay.

14. The apparatus of claim 7, wherein the plurality of sensors is selected from the group consisting of an acetone selective sensor, an ethanol selective sensor, and a methyl nitrate selective sensor.

15. The apparatus of claim 7, wherein the biomarker is selected from the group consisting of acetone, ethanol, and methyl nitrate.

16. A method for detecting hyperglycemia comprising:
(a) flowing a breath gas sample through a breath inlet positioned at an entrance of a housing, wherein the housing comprises a breath flow pathway disposed within the housing;
(b) exposing the breath gas sample to an acetone sensor element positioned in the breath flow pathway, downstream from the breath inlet;
(c) exposing the breath gas sample to an ethanol sensor element positioned in the breath flow pathway, downstream from the acetone sensor element;
(d) exposing the breath gas sample to a methyl nitrate sensor element positioned in the breath flow pathway, downstream from the ethanol sensor element; and
(e) releasing the breath gas sample through a breath outlet positioned at an exit of the breath flow pathway.

17. The method of claim 16, further comprising the step of controlling the humidity of the breath gas sample in at least a portion of the breath flow pathway.

18. The method of claim 16, further comprising the step of releasing at least a portion of the breath gas sample through an excess exhaust portal, before the breath gas sample is exposed to the acetone sensor element.

19. The apparatus of claim 16, wherein the ethanol selective sensor element comprises a zinc oxide material deposited on a gold microelectrode array and a catalyst material.

20. The method of claim 17, wherein the methyl nitrate selective sensor element comprises a micro-channel reactor filter comprising platinum and zeolite, and a potentiometric nitric oxide (NO) sensor.

21. The method of claim 20, wherein the methyl nitrate sensor element further comprises a micro-channel reactor filter heater relay.

22. A method for monitoring hyperglycemia comprising:
(a) flowing a breath gas sample through a breath inlet positioned at an entrance of a housing, wherein the housing comprises a breath flow pathway disposed within the housing;
(b) exposing the breath gas sample to a plurality of sensors positioned in the breath flow pathway, downstream from the breath inlet, wherein each sensor is configured to detect the presence of a biomarker indicative of hyperglycemia; and
(c) releasing the breath gas sample through a breath outlet positioned at an exit of the breath flow pathway.

23. The method of claim 22, further comprising the step of releasing a portion of the breath gas sample from the housing without contacting the plurality of sensors, through an excess exhaust portal.

24. The method of claim 22, further comprising the step of controlling the humidity of the breath gas sample in at least a portion of the breath flow pathway.

25. The method of claim 22, wherein the plurality of sensors comprises an acetone selective sensor, an ethanol selective sensor, and a methyl nitrate selective sensor.

26. The method of claim 22, wherein the ethanol selective sensor comprises a zinc oxide material deposited on a gold microelectrode array and a catalyst material.

27. The method of claim 22, wherein the methyl nitrate selective sensor comprises a sensor assembly, and the sensor assembly comprises a micro-channel reactor filter comprising platinum and zeolite, and a potentiometric nitric oxide (NO) sensor.

28. The method of claim 27, wherein the methyl nitrate selective sensor further comprises a micro channel reactor filter heater relay.

29. The method of claim 22, wherein the plurality of sensors is selected from the group consisting of an acetone selective sensor, an ethanol selective sensor, and a methyl nitrate selective sensor.

30. The method of claim 22, wherein the biomarker is selected from the group consisting of acetone, ethanol, and methyl nitrate.
